(19) United States
(12) Patent Application Publication
(10) Pub. No.: US 2008/0031843 A1
(43) Pub. Date: Feb. 7, 2008

(54) Soap-free, self-foaming shaving gel comprising an N-acylsarcosine

(75) Inventors: Lionnel Aubert, Asnieres Sur Oise (FR); Lydia Dussault, St Nom Le Breteche (FR)

Correspondence Address: BUCHANAN, INGERSOLL & ROONEY PC, POST OFFICE BOX 1404, ALEXANDRIA, VA, 22313-1404, US

(73) Assignee: L'Oreal, Paris (FR)

(21) Appl. No.: 11/819,057

(22) Filed: Jun. 25, 2007

Related U.S. Application Data
(60) Provisional application No. 60/819,654, filed on Jul. 11, 2006.

(30) Foreign Application Priority Data
Jun. 23, 2006 (FR) 0652622

(51) Int. Cl. A61K 8/30; A61Q 9/02

(52) U.S. Cl. 424/70.19

(57) ABSTRACT

Soap-free, Self-foaming shaving gels contain, in a cosmetically acceptable medium: a) at least one aqueous phase;
b) at least one N-acylsarcosine wherein the acyl radical is a C₁₀-C₂₀ radical in an amount less than 4 wt % relative to the total weight of the composition;
c) at least one mineral or organic base in an amount sufficient to dissolve the N-acylsarcosine and produce a pH of 4 to 8; d) at least one anionic surfactant; e) at least one non-ionic surfactant; and f) at least one self-foaming agent.

### CROSS-REFERENCE TO PRIORITY/PROVISIONAL APPLICATIONS

This application claims priority under 35 U.S.C. § 119 of FR 0652622, filed Jun. 23, 2006, and of U.S. Provisional Application No. 60/819,654, filed Jul. 11, 2006, each hereby expressly incorporated by reference and each assigned to the assignee hereof.

### BACKGROUND OF THE INVENTION

1. Technical Field of the Invention

The present invention relates to soap-free self-foaming shaving gels based on an N-acylsarcosine. Such compositions are distributed in the form of a gel containing a volatile compound that produces a gel which is converted into a mousse when it is spread onto the skin in preparation for wet-shaving using a razor.

1. Description of Background and/or Related and/or Prior Art

Shaving foams and self-foaming shaving gels coexist on the market for aerosol shaving products.

Self-foaming shaving gels are well known and have been described, for example, in U.S. Pat. Nos. 2,995,521; 3,541,581, 4,405,489; 4,528,111; 4,651,503; 5,248,495; 5,308,643; 5,326,556 and WO 91/07943. Such formulations are in the form of an oil-in-water emulsion in which the self-foaming agent, generally a volatile aliphatic hydrocarbon (i.e., having a low boiling point) is dissolved in the oily phase, and the aqueous phase comprises a water-soluble soap. The product is generally packaged in an aerosol container with a separation such as a piston or a flexible pocket to separate the self-foaming agent from the propellant necessary for ejecting the product. The product is applied in the form of a transparent, translucent or opaque gel which is substantially foam-free until it is spread onto the skin, at which time it produces a foam by evaporation of the volatile hydrocarbon foaming agent.

Conventional self-foaming shaving gels are valued by a wide range of consumers. However, these products, due to the presence of soap in the composition, have a tendency to dry out the skin and to increase the roughness of the skin. To mitigate this effect, self-foaming shaving gels are formulated with softening agents such as humectants, emollients, silicones, etc. The incorporation of these additives has an influence on the aesthetic qualities of the product and may also cause dryness of the skin by repeated use. For these reasons “soap-free” shaving gels containing N-acylsarcosinates have been developed.

N-acylsarcosinates are well-known anionic surfactants having the formula: 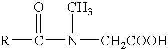
in which R is a fatty acid hydrocarbon-based chain. These surfactants are generally employed in the form of water-soluble salts formed by neutralizing with sodium hydroxide, potassium hydroxide, ammonium hydroxide or triethanolamine and have been recommended in a wide range of products such as shampoos, detergents, toothpastes, shaving creams or hand soaps. For example, aerosol shaving creams containing sarcosinates are described in U.S. Pat.s Nos. 3,959,160; 4,113,643 and 4,140,648 and also in the document Harry's Cosmetology (7th edition, 1982, page 169— see Croda Cosmetic and Pharmaceutical Formulary Supplement, formula SV11). A non-aerosol shaving cream possibly containing an N-acylsarcosinate has been described in U.S. Pat. No. 4,892,729 and a non-aerosol shaving gel containing a soap and a sarcosinate has been described in U.S. Pat. No. 5,340,571.

To overcome the aforementioned drawbacks, soap-free, self-foaming shaving gels comprising 65 to 85% of water, 4 to 16 wt % of N-acylsarcosine in which the acyl radical is a C₁₀-C₂₀ radical, an organic or mineral base in a sufficient amount to dissolve the N-acylsarcosine and produce a pH of 4 to 8, 1 to 8 wt % of a self-foaming agent and 1 to 10 wt % of a non-volatile paraffinic hydrocarbon fluid are proposed in EP-782,436.

In WO 05/094764 soap-free self-foaming shaving gels are proposed comprising 65 to 85% of water, 4 to 16 wt % of N-acylsarcosine in which the acyl radical is a C₁₀-C₂₀ radical, an organic or mineral base in a sufficient amount to dissolve the N-acylsarcosine and produce a pH of 4 to 8, 1 to 8 wt % of a self-foaming agent and 0.25 to 5 wt % of a C₃-C₆ short-chain polyol (glycerol, propylene glycol).

These formulae, on the one hand, require the presence of N-acylsarcosine at content levels greater than or equal to 4% in order to form a gel of sufficient stiffness for good use. On the other hand, the qualities of the foam obtained during the application to the skin still remain insufficient.

### SUMMARY OF THE INVENTION

It has now surprisingly been determined that it is possible to obtain self-foaming gels of acceptable stiffness and having good soap-free foaming properties without the aforementioned drawbacks by incorporating an N-acylsarcosine at content levels less than 4% in the presence of a mineral or organic base in a sufficient amount to dissolve the N-acylsarcosine and produce a pH of 4 to 8; at least one anionic surfactant, at least one non-ionic surfactant, and at least one self-foaming agent.

The present invention therefore features soap-free, self-foaming shaving gels comprising, formulated into a cosmetically acceptable medium:
a) at least one aqueous phase;
b) at least one N-acylsarcosine wherein the acyl radical is a C₁₀-C₂₀ radical in an amount less than 4 wt % relative to the total weight of the composition;
c) at least one mineral or organic base in an amount sufficient to dissolve the N-acylsarcosine and produce a pH of 4 to 8;
d) at least one anionic surfactant;
e) at least one non-ionic surfactant; and
f) at least one self-foaming agent.

The present invention also features a shaving method that entails applying a self-foaming shaving gel as defined above to the skin.

This invention also features a shaving kit that comprises:
(a) at least one self-foaming shaving gel as defined above; and
(b) at least one, especially disposable, razor; and/or
(c) a means of spreading a shaving composition.

### DETAILED DESCRIPTION OF BEST MODE AND SPECIFIC/PREFERRED EMBODIMENTS OF THE INVENTION

The term “soap-free” means containing less than 1% by weight of soap.

The term “cosmetically acceptable medium” means a medium that is compatible with the skin and/or its appendages, which has a pleasant color, odor and feel and which does not generate unacceptable discomfort (stinging, tautness or redness) liable to dissuade the consumer from using this composition.

The N-acylsarcosines according to the invention are preferably selected from those having a C₁₂-C₁₈ acyl radical. More preferentially, they are selected from among stearoyl sarcosine, myristoyl sarcosine, oleoyl sarcosine, lauroyl sarcosine, cocoyl sarcosine or mixtures thereof. Even more preferentially, they are selected from among stearoyl sarcosine, myristoyl sarcosine and mixtures thereof. The sarcosine or sarcosines are present at content levels less than 4 wt % and preferably ranging from 2.5 to 3.5% relative to the total weight of the composition.

According to one particular embodiment of the invention, it is possible to employ a pre-neutralized sarcosinate. In this case, it will not be necessary to add the base separately to the composition unless the pH of the composition has to be adjusted as desired.

The base may be selected from among mineral bases such as potassium hydroxide, sodium hydroxide or ammonium hydroxide. It may be selected from among organic bases, in particular alkanolamines such as isopropanolamine, mono-, di- and triethanolamine, aminoethylpropanol and aminomethylpropanol. Triethanolamine is preferred. The amount of base used depends on the amount of sarcosine present in the composition. A sufficient amount of base must be used to dissolve the sarcosine in the aqueous phase and produce a pH of 4 to 8, and more preferentially of 5 to 7. To reach this pH range, the sarcosine is preferably 50 to 90%, more preferentially 60 to 80%, neutralized. Preferably the sarcosine will be used in a slight molar excess relative to the base. The base is preferably present at a level varying from 1 to 6% relative to the total weight of the composition.

The aqueous phase of the compositions according to the invention preferably is from 65 to 85 wt %, and more preferentially from 70 to 80 wt %, of the total weight of the composition.

As anionic surfactants according to the invention, exemplary are carboxylates, oxyethylenated or non-oxyethylenated alkyl sulfates, sulfonates, alkyl sulfoacetates, phosphates, polypeptides, anionic derivatives of alkyl polyglucoside, and mixtures thereof.

As carboxylates, exemplary are:
amido ether carboxylates (AECs), such as sodium lauryl amido ether carboxylate (3 EO) marketed under the trademark AKYPO FOAM 30® by Kao Chemicals;
polyoxyethylenated carboxylic acid salts, such as oxyethylenated (6 EO) sodium lauryl ether carboxylate (65/25/10 C₁₂₋₁₄₋₁₆) marketed under the trademark AKYPO SOFT 45 NV® by Kao Chemicals; polyoxyethylenated and carboxymethylated fatty acids of olive oil, the product marketed under the trademark OLIVEM 400® by Biologia E Tecnologia; oxyethylenated (6 EO) sodium tridecyl ether carboxylate marketed under the trademark NIKKOL ECTD-6NEX® by Nikkol;
acetates, such as sodium 2-(2-hydroxyalkyloxy) acetate marketed under the trademark BEAULIGHT SH AA by Sanyo;
alaninates such as sodium N-lauroyl-N-methylamidopropionate, marketed under the trademark SODIUM NIKKOL ALANINATE LN 30® by Nikkol or under the trademark ALANONE ALE® by Kawaken, and triethanolamine N-lauroyl-N-methylalanine marketed under the trademark ALANONE ALTA® by Kawaken; (3) acylglutamates, such as triethanolamine monococoylglutamate marketed under the trademark ACYLGLUTAMATE CT-12® by Ajinomoto, and triethanolamine lauroylglutamate marketed under the trademark ACYLGLUTAMATE LT-12® by Ajinomoto; (4) aspartates, such as the mixture of triethanolamine N-lauroylaspartate and triethanolamine N-myristoylaspartate, marketed under the trademark ASPARACK® by Mitsubishi; (5) glycinates, such as sodium N-cocoylglycinate marketed under the trademarks AMILITE GCS-12® and AMILITE GCK 12 by Ajinomoto;
citrates, such as the oxyethylenated (9 EO) citric monoester of coco alcohols marketed under the trademark WITCONOL EC 1129 by Goldschmidt; and
galacturonates, such as sodium dodecyl-D-galactoside uronate marketed by Soliance.

As oxyethylenated or non-oxyethylenated alkyl sulfates, exemplary are (2.2 EO) sodium lauryl ether sulfate (70/30 C₁₂₋₁₄) marketed under the trademark SIPON AOS 225® by Cognis, (3 EO) ammonium lauryl ether sulfate (70/30 C₁₂₋₁₄) marketed under the trademark SIPON LEA 370® by Cognis, (9 EO) ammonium alkyl (C₁₂-C₁₄) ether sulfate marketed under the trademark RHODAPEX AB/20® by Rhodia Chimie, and the mixture of sodium and magnesium lauryl and oleyl ether sulfate marketed under the trademark EMPICOL BSD 52 by Albright & Wilson.

As sulfonates, exemplary are (1) α-olefin sulfonates, such as sodium α-olefin (C₁₄₋₁₆) sulfonate marketed under the trademark BIOTERGE AS-40® by Stepan, under the trademarks WITCONATE AOS PROTEGE® and SULFRAMINE AOS PH 12® by Witco, the sodium secondary olefin sulfonate marketed under the trademark HOSTAPUR SAS 30® by Clariant; (2) isethionates, such as sodium cocoylisethionate, such as the product marketed under the trademark JORDAPON CI P® by Jordan; and (3) taurates, such as the sodium salt of palm kernel oil methyltaurate marketed under the trademark HOSTAPON CT PATE® by Clariant; N-acyl N-methyltaurates, such as sodium N-cocoyl N-methyltaurate marketed under the trademark HOSTAPON LT-SF® by Clariant or marketed under the trademark NIKKOL CMT-30-T® by Nikkol, sodium palmitoyl methyltaurate marketed under the trademark NIKKOL PMT® by Nikkol.

As sulfosuccinates, exemplary are oxyethylenated (3 EO) lauryl alcohol monosulfosuccinate (70/30 C₁₂/C₁₄) marketed under the trademarks SETACIN 103 SPECIAL®, REWOPOL SB-FA 30 K 4® by Witco, the disodium salt of a C₁₂-C₁₄ alcohol hemisulfosuccinate marketed under the trademark SETACIN F SPECIAL PASTE® by Zschimmer Schwarz, oxyethylenated (2 EO) disodium oleamidosulfosuccinate marketed under the trademark STANDAPOL SH 135® by Cognis, oxyethylenated (5 EO) laurylamide monosulfosuccinate marketed under the trademark LEBON A-5000® by Sanyo.

As phosphates, exemplary are monoalkyl phosphates and dialkyl phosphates, such as lauryl monophosphate marketed under the trademark MAP 20® by Kao Chemicals, the potassium salt of dodecylphosphoric acid, as a mixture of monoester and diester (mainly diester) marketed under the trademark CRAFOL AP-31® by Cognis, the mixture of octylphosphoric acid monoester and diester marketed under the trademark CRAFOL AP-20® by Cognis, and the mixture of ethoxylated (7 mol of EO) 2-butyloctanol phosphoric acid monoester and diester marketed under the trademark ISOFOL 12 7 EO-PHOSPHATE ESTER® by Condea.

As polypeptides (which are compounds obtained by condensation of a fatty chain over cereal, and especially wheat and oat, amino acids), exemplary are the potassium salt of hydrolyzed lauroyl wheat protein, marketed under the trademark AMINOFOAM W OR® by Croda; the triethanolamine salt of hydrolyzed cocoyl soya bean protein, marketed under the trademark MAY-TEIN SY® by Maybrook; the sodium salt of lauroyl oat amino acids, marketed under the trademark PROTEOL OAT® by Seppic; the hydrolysate of collagen grafted onto coconut fatty acid marketed under the trademark GELIDERM 3000® by Deutsche Gelatine; and soya bean proteins acylated with hydrogenated coconut acids, marketed under the trademark PROTEOL VS 22® by Seppic.

As anionic alkyl polyglucoside derivatives, exemplary are the glyceryl citrates, tartrates, sulfosuccinates, carbonates and ethers obtained from alkyl polyglucosides. Also exemplary are the sodium salt of cocoyl polyglucoside (1.4) tartaric ester, marketed under the trademark EUCAROL AGE-ET® by Cesalpinia; the disodium salt of cocoyl polyglucoside (1.4) sulfosuccinic ester, marketed under the trademark ESSAI 512 MP® by Seppic; the sodium salt of cocoyl polyglucoside (1.4) citric ester, marketed under the trademark EUCAROL AGE-EC® by Cesalpinia, and the sodium lauryl polyglucoside ether carboxylate marketed under the trademark PLANTAPON LGC SORB by Cognis.

Preferably, the salts of C₆-C₂₄ alkyl ether sulfates having 1 to 30 ethylene oxide groups will be used, in particular the alkali metal or alkaline-earth metal, ammonium, amine or amino alcohol salts and more particularly the sodium salts and even more particularly oxyethylenated sodium (C₁₂-C₁₄) alkyl ether sulfates having an average number of ethylene oxide groups from 1 and 4, and more particularly sodium laureth sulfate (CTFA name).

The compositions according to the invention comprise one or more non-ionic surfactants. These are compounds that are well known per se (in this respect see, in particular, “Handbook of Surfactants” by M. R. Porter, published by Blackie & Son (Glasgow and London), 1991, pp. 116-178). Thus they can especially be selected from among fatty alcohols having a fatty chain that preferably comprises 8 to 20 carbon atoms; polyethoxylated, polypropoxylated or polyglycerolated alcohols, α-diols, alkylphenols or fatty acids, having a fatty chain comprising, preferably, 8 to 20 carbon atoms, and where the number of ethylene oxide groups or propylene oxide groups varies preferably from 2 to 60 and the number of glycerol groups optionally ranging especially from 2 to 30. Also exemplary are the copolymers of ethylene and propylene oxide, the condensates of ethylene and propylene oxide with fatty alcohols; polyethoxylated fatty amides preferably having from 2 to 30 mol of ethylene oxide, polyglycerolated fatty amides preferably comprising, on average, 1 to 5, and in particular 1.5 to 4, glycerol groups; polyethoxylated fatty amines preferably having 2 to 30 mol of ethylene oxide; ethoxylated sorbitan fatty acid esters preferably having 2 to 30 mol of ethylene oxide; sucrose fatty acid esters, polyethylene glycol fatty acid esters, (C₆-C₂₄)alkyl polyglycosides, N-(C₆-C₂₄)alkylglucamine derivatives, amine oxides such as (C₁₀-C₁₄)alkylamine oxides or N-(C₁₀-C₁₄)acylaminopropylmorpholine oxides; and mixtures thereof.

As alkyl polyglucosides, preferably those having an alkyl group comprising 6 to 30 carbon atoms and preferably 8 to 16 carbon atoms, and having a hydrophilic group (glucoside) preferably comprising 1.2 to 3 saccharide units are used. As alkyl polyglucosides, exemplary are decyl glucoside (C₉/C₁₁ alkyl polyglucoside (1.4)) such as the product marketed under the trademark MYDOL 10® by Kao Chemicals, under the trademark PLANTAREN 2000 UP® by Cognis, and under the trademark ORAMIX NS 10® by Seppic; caprylyl/capryl glucoside, such as the product marketed under the trademark ORAMIX CG 110® by Seppic; lauryl glucoside, such as the products marketed under the trademarks PLANTAREN 1200 N® and PLANTACARE 1200® by Cognis; and coco glucoside, such as the product marketed under the trademark PLANTACARE 818/UP® by Cognis.

The maltose derivatives are, for example, those described in EP-A-566,438, such as O-octanoyl-6′-D-maltose, or else O-dodecanoyl-6′-D-maltose described in FR-2,739,556.

Among the polyglycerolated fatty alcohols, exemplary is polyglycerolated (3.5 mol of glycerol) dodecanediol, a product manufactured under the trademark CHIMEXANE NF® by Chimex.

The preferred non-ionic surfactants are selected from among:
fatty alcohols having a C₈-C₂₀ fatty chain, more preferentially having a C₁₂-C1₁₈ fatty chain, such as for example myristyl alcohol, lauryl alcohol, stearyl alcohol and octyldodecanol;
polyoxyethylenated ethers of fatty alcohols having a C₈-C₂₀ fatty chain, more preferentially having a C₁₂-C₁₈ fatty chain and having 2 to 60, more preferentially 2 to 30 ethylene oxide units. Among these compounds, exemplary are Oleth-20, Steareth-21, Ceteth-20, Laureth-4 and Laureth-23. More preferentially, a mixture of Laureth-4 and Laureth-23 will be selected.

The non-ionic surfactant or surfactants are preferably present at concentrations ranging from 5 to 20 wt % and more preferentially from 7 to 12 wt % relative to the total weight of the composition.

The self-foaming agent is preferably selected from among volatile hydrocarbons and halogenated volatile hydrocarbons having a sufficiently low boiling point to make it possible for the latter to evaporate and cause the gel to foam on application to the skin, and a sufficiently high boiling point to avoid producing a foam prematurely. The boiling point of the self-foaming agent preferably varies from −20 to 40°C. The self-foaming agent is preferably selected so as to form a vapor pressure at 20°C of3 to 20 psig and preferably of 5 to 15 psig. The self-foaming agents that can be used according to the invention are selected from among C₄-C₆ aliphatic hydrocarbons such as n-pentane, isopentane, neopentane, n-butane, isobutane and mixtures thereof. More preferentially, an isopentane/isobutane mixture will be used in a weight ratio ranging from 1/1 to 3/1. The self-foaming agent is preferably present at concentrations ranging from 1 to 8 wt % and more preferentially from 2 to 5 wt % relative to the total weight of the composition.

The compositions of the invention may comprise, in addition, a gelling agent and, for example, comprise at least one non-volatile liquid hydrocarbon. The terms “volatile” and “liquid” signify that these materials are liquid at room temperature and have a boiling point above 200° C. Among these liquid hydrocarbons, exemplary are mineral oils, and branched aliphatic liquids. These liquids preferably have 16 to 48 carbon atoms, more preferentially 20 to 40 carbon atoms and a kinetic viscosity (measured according to the ASTM D445 standard) of 5 to 100 cst and more preferentially of 10 to 70 cst at 40° C. The preferred non-volatile liquid hydrocarbons are selected from among mineral oils having a kinetic viscosity of 10 to 70 cst, hydrogenated polyisobutenes having a molecular weight from 320 to 420, and mixtures thereof. The non-volatile liquid hydrocarbon or hydrocarbons are preferably present at concentrations less than or equal to 10% and preferably less than or equal to 7% by weight relative to the total weight of the composition.

The compositions of the invention may also comprise an auxiliary water-soluble gelling agent or a thickening agent to improve the consistency and the stability of the gel or to adjust the viscosity.

Among these auxiliary gelling agents, exemplary are the hydroxyalkyl cellulose polymers such as hydroxyethyl cellulose or hydroxypropyl cellulose (products marketed respectively under the trademark NATROSOL or KLUCEL); acrylic acid/polyallyl sucrose copolymers (products marketed under the trademark CARBOPOL); carboxymethyl cellulose and methyl ether cellulose (products marketed under the trademark METHOCEL); natural or synthetic gums, or starches. The preferred thickening agents are selected from among hydroxyethyl cellulose or hydroxypropyl cellulose or mixtures thereof. The auxiliary gelling agents or thickening agents are preferably present at concentrations ranging from 0.01 to 5 wt %, more preferentially from 0.05 to 2 wt % and even more preferentially from 0.01 to 2 wt %, relative to the total weight of the composition.

The compositions of the invention may contain, in addition, a short-chain polyol to improve the foam qualities and/or stability of the composition. The polyol or polyols are preferably present at concentrations less than 5 wt % and more preferentially ranging from 0.25 to 5 wt % relative to the total weight of the composition. Among the polyols that can be used, exemplary are glycerol, propylene glycol or mixtures thereof.

The shaving compositions according to the invention may comprise, in addition, a variety of conventional cosmetic ingredients to improve the aesthetic qualities and performance of these compositions.

The compositions according to the invention may also comprise, in addition, a cationic conditioning polymer to improve the lubricity and feel of the skin after shaving. Exemplary are the quaternary ammonium salts of hydroxyethyl cellulose, such as Polyquaternium-10 or Polyquaternium-24.

The following cationic polymers are also exemplary:
Polyquaternium 5, such as the product MERQUAT 5 marketed by Calgon;
Polyquaternium 6, such as the product SALCARE SC 30 marketed by Ciba, and the product MERQUAT 100 marketed by Calgon;
Polyquaternium 7, such as the products MERQUAT S, MERQUAT 2200 and MERQUAT 550 marketed by Calgon, and the product SALCARE SC 10 marketed by Ciba;
Polyquaternium 11, such as the products GAFQUAT 755, GAFQUAT 755N and GAFQUAT 734 marketed by ISP;
Polyquaternium 15, such as the product ROHAGIT KF 720 F marketed by Rohm;
Polyquaternium 16, such as the products LUVIQUAT FC905, LUVIQUAT FC370, LUVIQUAT HM552 and LUVIQUAT FC550 marketed by BASF;
Polyquaternium 22, such as the product MERQUAT 280 marketed by Calgon;
Polyquaternium 28, such as the product STYLEZE CC10 marketed by ISP;
Polyquaternium 39, such as the product MERQUAT PLUS 3330 marketed by Calgon;
Polyquaternium 44, such as the product LUVIQUAT CARE marketed by BASF;
Polyquaternium 46, such as the product LUVIQUAT HOLD marketed by BASF; and
Polyquaternium 47, such as the product MERQUAT 2001 marketed by Calgon.

It is also possible to use, as the cationic polymer, cationic guars such as the product JAGUAR marketed by Rhodia.

The cationic conditioning polymer or polymers are preferably present at concentrations ranging from 0.05 to 2 wt %, more preferentially ranging from 0.1 to 1 wt %, relative to the total weight of the composition.

Other additives may also be included in the compositions of the invention, such as:
humectants such as sorbitol;
emollients such as fatty esters like isopropyl myristate, decyl oleate, 2-ethyhexyl palmitate, PEG-7 glyceryl cocoate and glyceryl linoleate; propoxylated fatty ethers such as PPG-10 cetyl ether and PPG-11 stearyl ether; diglycerides or triglycerides such as lecithin, the mixture of capric/caprylic triglycerides, PEG-10 soy sterol or vegetable oils;
refreshing agents and soothing agents such as menthol, aloe, allantoin, lanolin, bisabolol or hyaluronic acid;
lubricants such as polyethylene glycols (i.e., PEG-14M, PEG-23M), fluorosurfactants, silicones (i.e., dimethicone, dimethiconol, dimethicone copolyol, stearyl dimethicone, cetyl dimethicone copolyol, cyclomethicone, etc.);
vitamins, including precursors and derivatives such as panthenol, tocopheryl acetate, niacinamide, retinyl palmitate or vitamin A palmitate;
colorants;
fragrances;
antioxidants;
anti-bacterial and/or anti-fungal agents; and
preservatives (i.e., methylchloroisothiazolinone, methylisothiazolinone, DMDM hydantoin, iodopropynyl butylcarbamate).

Of course, one skilled in this art will take care to select the aforementioned optional additional compound or compounds and/or their amounts so that the advantageous properties intrinsically associated with the compositions conforming to the invention are not, or are not substantially, changed by the envisaged addition or additions.

The shaving compositions of the present invention may be packaged in any device enabling a self-foaming gel to be dispensed. For example, the device may be an aerosol container with a separation such as a piston or a flexible pocket to separate the self-foaming agent from the propellant that is necessary for ejecting the product. The device may also be a flexible tube; a pump dispenser or a dispenser having deformable walls.

The present invention also features a shaving kit that comprises:
a) at least one self-foaming shaving gel as defined above; and
b) at least one, especially disposable, razor; and/or
c) a means of spreading a shaving composition.

According to one particular embodiment of the invention, the kit may contain, in addition, an after-shave composition to soothe razor burn.

In order to further illustrate the present invention and the advantages thereof, the following specific examples are given, it being understood that same are intended only as illustrative and in nowise limitative. In said examples to follow, all parts and percentages are given by weight, unless otherwise indicated. The names of the compounds are the chemical names or the CTFA (International Cosmetic Ingredient Dictionary and Handbook) names, as appropriate.

### EXAMPLE

The following soap-free, self-foaming shaving gels A and B (not according to the invention) were prepared, which were in bag valves.

| | | Composition B |
|---|---|---|
| Ingredients | Composition A | (outside the invention) |
| BHT | 0.07 g | 0.07 g |
| Copper gluconate | 0.01 g | 0.01 g |
| Triethanolamine | 0.05 g | 0.05 g |
| Bisabolol | 1.16 g | 1.16 g |
| 2,6-dimethyl-7-octen-22-ol | 0.39 g | 0.39 g |
| Paraffinum Liquidum | 0.96 g | 0.96 g |
| Polyquaternum-10 | 0.09 g | 0.09 g |
| Hydroxypropyl cellulose | 0.09 g | 0.09 g |
| Isopentane and Isobutane | 3.50 g | 3.50 g |
| (75/25) | | |
| Glycerol | 3.86 g | 3.86 g |
| Stearoyl sarcosine (and) Myti- | 2.90 g | 2.90 g |
| stoyl sarcosine (75%/25%) | | |
| Laureth-23 | 6.75 g | 6.75 g |
| Myristyl alcohol | 2.41 g | 2.41 g |
| PEGG-14M | 0.19 g | 0.19 g |
| (Polyethylene Glycol 14 000 | | |
| EO) | | |
| Laureth-4 | 0.48 g | 0.48 g |
| 70% 2.2 EO sodium laureth | 7.72 g | — |
| sulphate in aqueous solution | | |
| Water | qs for 100 g | qs for 100 g |
| Stiffness of the gel (in grams) | 23 g | 2.5 g |

The stiffness of each gel was measured at 25° C. using a TA XT2i texture analyzer manufactured by Thermo equipped with an SMS P/0-5 HS cylinder 0.5 inch diameter hemi-spherical delrin cylinder probe. The stiffness (expressed in grams) of each product in compression was measured by said cylinder at a rate of 2 mm/s over a distance of 25 mm. It was observed that the composition B from the prior art that did not contain any anionic surfactant and that contained less than 4 wt % of sarcosine made it impossible to obtain a stiff gel unlike composition A of the invention that contained an anionic surfactant.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A soap-free, self-foaming shaving gel comprising:
a) at least one aqueous phase;
b) at least one N-acylsarcosine where the acyl radical is a C₁₀C₂₀ radical in an amount less than 4 wt % relative to the total weight of the composition;
c) at least one mineral or organic base in an amount sufficient to dissolve the N-acylsarcosine and produce a pH of 4 to 8;
d) at least one anionic surfactant;
e) at least one non-ionic surfactant; and
f) at least one self-foaming agent, formulated into a cosmsetically acceptance medium therefor.

2. The shaving gel as defined by claim 1, wherein the N-acylsarcosine has a C₁₂C₁₈ acyl radical.

3. The shaving gel as defined by claim 1, wherein the N-acylsarcosine is selected from the group consisting of stearoyl sarcosine, myristoyl sarcosine, oleoyl sarcosine, lauroyl sarcosine, cocoyl sarcosine and mixtures thereof.

4. The shaving gel as defined by claim 3, wherein the N-acylsarcosine is selected from among stearoyl sarcosine, myristoyl sarcosine and mixtures thereof.

5. The shaving gel as defined by claim 1, wherein the N-acylsarcosine or N-acylsarcosines are present at content levels ranging from 2.5% to 3.5% relative to the total weight of the composition.

6. The shaving gel as defined by claim 1, wherein the base is an alkanolamine.

7. The shaving gel as defined by claim 6, wherein the base is triethanolamine.

8. The shaving gel as defined by claim 1, wherein the sarcosine is 50% to 90% neutralized.

9. The shaving gel as defined by claim 1, wherein the base is present at a level varying from 1% to 6% relative to the total weight of the composition.

10. The shaving gel as defined by claim 1, wherein the aqueous phase is from 65% to 85 wt % of the total weight of the composition.

11. The shaving gel as defined by claim 1, wherein the anionic surfactant is selected from the group consisting of carboxylates, oxyethylenated or non-oxyethylenated alkyl sulfates, sulfonates, alkyl sulfoacetates, phosphates, polypeptides, anionic derivatives of alkyl polyglucoside, and mixtures thereof.

12. The shaving gel as defined by claim 11, wherein the anionic surfactant is selected from the groups consisting of the salts of C₆-C₂₄ alkyl ether sulfates having 1 to 30 ethylene oxide groups and the oxyethylenated sodium salts of C₁₂C₁₄ alkyl ether sulfates having an average number of ethylene oxide groups from 1 and 4.

13. The shaving gel as defined by claim 1, wherein the non-ionic surfactant is selected from the group consisting of fatty alcohols; polyethoxylated, polypropoxylated or polyglycerolated alcohols, α-diols, alkylphenols or fatty acids; the copolymers of ethylene and propylene oxide; the condensates of ethylene and propylene oxide with fatty alcohols; polyethoxylated fatty amides; polyglycerolated fatty amides; polyethoxylated fatty amines; ethoxylated sorbitan fatty acid esters; sucrose fatty acid esters, polyethylene glycol fatty acid esters; (C₆C₂₄)alkyl polyglycosides; N-(C₆-C₂₄) alkylglucamine derivatives; amine oxides and mixtures thereof.

14. The shaving gel as defined by claim 13, wherein the non-ionic surfactant or surfactants are selected from among:
fatty alcohols having a C₈-C₂₀ fatty chain;
polyoxyethylenated ethers of fatty alcohols having a C₈-C₂₀ fatty chain, and having 2 to 60 ethylene oxide units, and mixtures thereof.

15. The shaving gel as defined by claim 1, wherein the non-ionic surfactant or surfactants are present at concentrations ranging from 5 to 20 wt % relative to the total weight of the composition.

16. The shaving gel as defined by claim 1, wherein the self-foaming agent is selected from volatile hydrocarbons and halogenated volatile hydrocarbons, having a boiling point ranging from −20 to 40° C.

17. The shaving gel as defined by claim 16, wherein the self-foaming agent is a C₄-C₆ aliphatic hydrocarbon.

18. The shaving gel as defined by claim 17, wherein the self-foaming agent is selected from the group consisting of n-pentane, isopentane, neopentane, n-butane, isobutane and mixtures thereof.

19. The shaving gel as defined by claim 18, wherein the self-foaming agent is an isopentane/isobutane mixture in a weight ratio ranging from 1/1 to 3/1.

20. The shaving gel as defined by claim 1, wherein the self-foaming agent is present at concentrations ranging from 1 to 8 wt % relative to the total weight of the composition.

21. The shaving gel as defined by claim 1, further comprising at least one gelling agent.

22. The shaving gel as defined by claim 21, wherein the gelling agent is a non-volatile liquid hydrocarbon.

23. The shaving gel as defined by claim 22, wherein the gelling agent is selected from the group consisting of mineral oils, branched aliphatic liquids having 16 to 48 carbon atoms and a kinetic viscosity of 5 to 100 cst at 40° C.

24. The shaving gel as defined by claim 22, wherein the non-volatile liquid hydrocarbon or hydrocarbons are present at concentrations less than or equal to 10% by weight relative to the total weight of the composition.

25. The shaving gel as defined by claim 1, further comprising at least one auxiliary water-soluble gelling agent and/or at least one thickening agent.

26. The shaving gel as defined by claim 25, wherein the auxiliary water-soluble gelling agent or the thickening agent is selected from the group consisting of hydroxyalkyl cellulose polymers; acrylic acid/polyallyl sucrose copolymers; carboxymethyl cellulose and methyl ether cellulose; natural or synthetic gums; or starches.

27. The shaving gel as defined by claim 25, wherein the auxiliary water-soluble gelling agent and/or the thickening agent are present at concentrations ranging from 0.01 to 5 wt % relative to the total weight of the composition.

28. The shaving gel as defined by claim 1, further comprising at least one short-chain polyol.

29. The shaving gel as defined by claim 28, wherein the polyol or polyols are present at concentrations less than 5 wt % relative to the total weight of the composition.

30. The shaving gel as defined by claim 28, wherein the polyol or polyols are selected from among glycerol, propylene glycol or mixtures thereof.

31. The shaving gel as defined by claim 1, further comprising at least one additive selected from the group consisting of:
cationic conditioning polymers;
humectants;
emollients;
refreshing agents and soothing agents;
lubricants;
vitamins including precursors and derivatives;
colorants;
fragrances;
antioxidants;
anti-bacterial and/or anti-fungal agents; and
preservatives.

32. A shaving method which comprises applying a self-foaming shaving gel as defined by claim 1 to the surface of the skin to be shaved, then in shaving the hairs using a razor.

33. A shaving kit that comprises:
a) at least one self-foaming shaving gel as defined in claim 1;
b) at least one, optionally disposable razor; and/or
c) a means of spreading a shaving composition.

34. The shaving kit as defined by claim 33, further comprising an after-shave composition to soothe razor burn.
